(19) United States
(12) Patent Application Publication
(10) Pub. No.: US 2003/0082594 A1
(43) Pub. Date: May 1, 2003

(54) System and method for analyzing resistance of a pathogen to one or more treatments

(76) Inventors: Christopher J. Gabe, Oakville (CA); Robert M. Lloyd, Suwanee, GA (US); Kelly N. Dyer, North York (CA)

Correspondence Address: Torys LLP, Suite 3000, 79 Wellington St. West, Toronto, ON, M5K 1N2, CA

(21) Appl. No.: 10/212,710

(22) Filed: Aug. 7, 2002

Related U.S. Application Data
(60) Provisional application No. 60/311,068, filed on Aug. 8, 2001.

(51) Int. Cl. C12Q 1/68; G06F 19/00; G01N 33/48; G01N 33/50

(52) U.S. Cl. 435/6; 702/20

(57) ABSTRACT

A system and method identifies levels of resistance for various treatments by matching differences between a pathogen and a target pathogen to one or more criteria in each of one or more rules corresponding to a level of resistance for one or more treatments. A report is provided containing the highest level of resistance indicated for each treatment for which a level of resistance was identified.

### COPYRIGHT AUTHORIZATION

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention is related to computer software and more specifically to bioinformatic computer software.

### BACKGROUND OF THE INVENTION

When treating a patient for a pathogen, different treatments for that pathogen may have different levels of effectiveness. For some treatments, the pathogen or patient will be resistant to the treatment, possibly resistant to the treatment, partially resistant to the treatment or not resistant to the treatment. Where more than one treatment can be selected for a particular pathogen, selecting the most effective treatment can involve avoiding treatments for which the pathogen or patient will be most resistant or where other undesirable results, such as toxic effects, may occur. Conventional methods of determining resistance involve applying a treatment to a patient and determining whether the treatment is effective. If not, another treatment may be selected. Other methods involve applying this technique to more than one treatment at a time.

Because discontinuing applied treatments to rapidly mutating organisms such as the HIV virus can induce certain resistances, it can be desirable to determine in advance which treatments will be most effective. Prior art methods for determining effectiveness of treatments involve performing in vitro experiments on the virus, but such experiments can take too much valuable time, and so faster ways of determining effectiveness of treatments have been desired.

Resistance to a treatment can be a function of the state of certain nucleic acids, such as DNA, RNA or other biological sequences, at a specific location. As used herein, a “nucleic acid” is or includes a deoxyribonuclotide or ribonucleotide polymer in either a single- or double-stranded form and can encompass known analogs of natural nucleotides that can function in a manner similar to naturally occurring nucleotides. For example, the resistance of drugs used to treat patents with HIV can be a function of the presence of mutations from a target nucleic acid at certain locations on the nucleic acid of the virus itself. The resistance to individual drugs for a patient whose virus exhibits these mutations can be identified using the results of published research and noted for use by a care provider who selects the treatment or treatments.

However, in the case of HIV, a care provider is required to identify the mutations and then check all of the available research to determine the treatments to which the virus will be resistant, partially resistant or possibly resistant, a time consuming and error-prone process. This is because of the complexity of the rules for determining resistance or possible resistance to the myriad of drugs available to the care provider. For example, the virus may be resistant to a drug if two mutations of the virus nucleic acid are found at two particular locations along with at least one out of three mutations at three other locations. Because of the complexity of the rules and the growing number of drugs available for treating HIV, manual application of resistance data to identified HIV mutations using available research would be too prone to error and time consuming to be useful.

Another difficulty of identifying resistance to treatment from available research arises because the result of one study may identify one criteria, such as a set of one or more mutations, that may be associated with possible resistance to a drug, which could be an acceptable result, while the result of another study may indicate another condition such as a different set of one or more mutations that may cause resistance to the same drug, which would be unacceptable: in other words one or more rules may be superceded or negated by other rules. Thus, even with a list of all of the mutations of a pathogen, and all of the research summarizing each research result, application of the mutations to the set of rules can be an error-prone task because it may identify a treatment as both acceptable and not acceptable. If the care provider makes the identification of the drug as acceptable, but misses the identification of the drug as unacceptable, the wrong treatment may be selected. As a result, merely providing a list of the mutations of a pathogen and results of research might not improve the accuracy of the care provider in selecting the proper treatment.

Tables of results of studies can help identify resistance of certain treatments, but the rules may be too complex for application via a table, and different levels of resistance (e.g. resistant, possibly resistant or not resistant) to the treatment would again make even the use of the table a time-consuming and error prone process, especially in light of the relatively large number of treatments that may be available.

Even if an easy to use table of rules were formulated, a care provider may wish to use his judgment in formulating a treatment program for the pathogen. A care provider may have had success using rules from a particular type of research, such as large scale, independent clinical trials, but may not wish to trust rules that were developed from another type of research such as in vitro data unless results of the trusted research indicate that available treatments for which such trusted research has been carried out will not effective for the pathogen or patient. Thus, application of rules from tables is made more complex by the various types of research that can be used to generate the rules.

Because the research for a pathogen may not be complete, a caregiver may wish to have access to the judgment of experts. Experts can use their judgement and experience to make educated guesses about treatments. If available treatments are in short supply, even educated guesses may be valuable to the care provider where other treatments supported by research are either not effective or not feasible for the patient.

What is needed is a system and method that can assist a care provider in selecting one or more treatments according to the research supporting the selection of treatments and that can include treatment information arising from judgements made by experts.

### SUMMARY OF INVENTION

A system and method identifies at least one level of resistance expected for each of several treatments for a pathogen by matching differences between a nucleic acid (for example, the RNA of a patient's HIV virus) and a hypothetical target nucleic acid to rules indicating an expected level of resistance of one or more treatments. The system and method can provide, for each treatment, an indication of the highest level of resistance indicated by all of the rules matching the differences exhibited by the nucleic acid, so as to allow the care provider to determine the expected level of resistance of the pathogen to that treatment, as well as providing the other levels of resistance identified to allow the care provider to use his or her judgement in disregarding the highest level of resistance and selecting another expected level of resistance from those provided, for example by using supplemental information. The system and method can provide such supplemental information, such as an explanation of the type of research that identified each level of resistance identified for the treatment, to allow the care provider a way of selecting a level of resistance from among those provided. The types of research may include an extrapolation of data from research related to the treatment or the differences, allowing for the use of rules developed using the judgement of experts, even in the absence of directly related, completed research. The differences between the nucleic acid and the target nucleic acid may include the locations of differences, such as mutations of biological sequences. Pathogens may include bacteria or viruses, such as HIV, or a mixture of bacteria and viruses. Treatments may include any antiviral compound where polymorphic changes in genomic content occurs, including reverse transcriptase inhibitors or protease inhibitors, at least ten reverse transcriptase/protease inhibitors or at least ten treatments of any kind, allowing the present invention to be used where a relatively large number of treatments are available. The system and method can supply a default level of resistance for each treatment for which none of the differences match criteria for any rules corresponding to that treatment. The default level of resistance for each treatment may be determined using the number of available rules that correspond to that treatment.

### BRIEF DESCRIPTION OF THE DRAWINGS

FIG. 1 is a block schematic diagram of a conventional computer system.

FIG. 2 is a block schematic diagram of a system for analyzing resistance of a pathogen to one or more treatments according to one embodiment of the present invention.

FIG. 3 is a flowchart illustrating a method of analyzing resistance of a pathogen to one or more treatments according to one embodiment of the present invention.

### DETAILED DESCRIPTION OF A PREFERRED EMBODIMENT

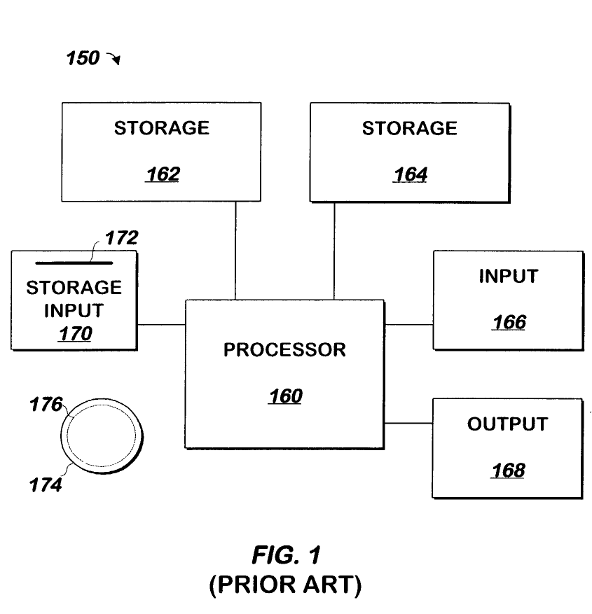

The present invention may be implemented as computer software on a conventional computer system. Referring now to FIG. 1, a conventional computer system 150 for practicing the present invention is shown. Processor 160 retrieves and executes software instructions stored in storage 162 such as memory, which may be Random Access Memory (RAM) and may control other components to perform the present invention. Storage 162 may be used to store program instructions or data or both. Storage 164, such as a computer disk drive or other nonvolatile storage, may provide storage of data or program instructions. In one embodiment, storage 164 provides longer term storage of instructions and data, with storage 162 providing storage for data or instructions that may only be required for a shorter time than that of storage 164. Input device 166 such as a computer keyboard or mouse or both allows user input to the system 150, and may include other means of input such as voice recognition. Output 168, such as a display or printer, allows the system to provide information such as instructions, data or other information to the user of the system 150. Storage input device 170 such as a conventional floppy disk drive or CD-ROM drive accepts via input 172 computer program products 174 such as a conventional floppy disk or CD-ROM or other nonvolatile storage media that may be used to transport computer instructions or data to the system 150. Computer program product 174 has encoded thereon computer readable program code devices 176, such as magnetic charges in the case of a floppy disk or optical encodings in the case of a CD-ROM which are encoded as program instructions, data or both to configure the computer system 150 to operate as described below.

In one embodiment, each computer system 150 is a conventional Sun Microsystems Ultra 10 workstation running the Solaris operating system commercially available from Sun Microsystems of Palo Alto, Calif., a Pentium-compatible personal computer system such as are available from Dell Computer Corporation of Round Rock, Tex. running the Windows (95, 98, ME, NT or XP) operating system commercially available from Microsoft Corporation of Redmond Wash. or a Macintosh computer system running the MacOS or OpenStep operating systems, commercially available from Apple Computer Corporation of Cupertino, Calif. and the Netscape browser commercially available from Netscape Computer Corporation of Mountain View, Calif. although other systems may be used.

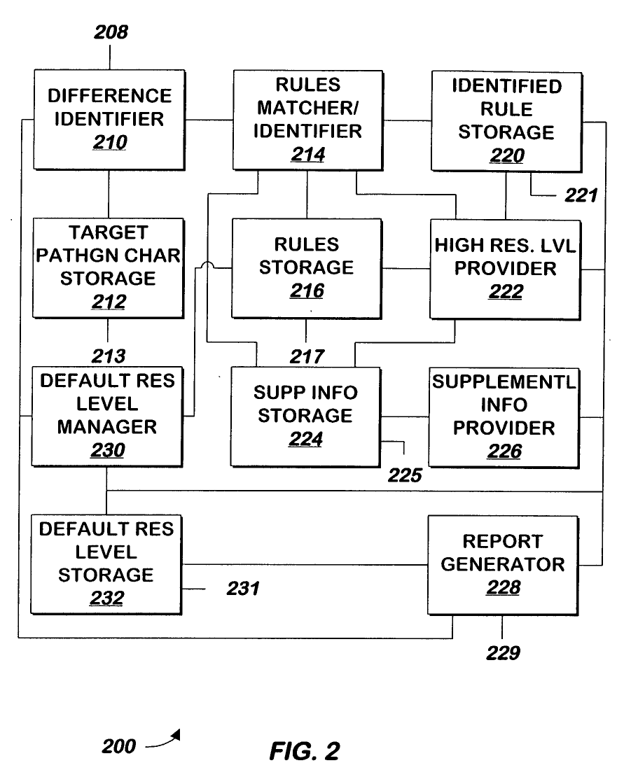

Referring now to FIG. 2, a system 200 for analyzing resistance by a pathogen to one or more treatments is shown according to one embodiment of the present invention. The system 200 is described herein as applied to the HIV virus, but could be used to determine resistance to treatment of any virus or bacteria or other pathogen.

The system 200 receives at input 208 one or more characteristics of a nucleic acid (as defined above), which may be a nucleic acid of the pathogen, a nucleic acid of the patient with the pathogen being treated, a nucleic acid of a person from whom the patient received the pathogen, or any other nucleic acid. Although the nucleic acid is described below as the nucleic acid of the pathogen itself, the description below is intended to be a representative sample of one embodiment of the present invention, and any other type of nucleic acid may be used by the present invention.

In one embodiment, the characteristics received at input 208 are nucleic acid sequences such as DNA or RNA sequences or other biological sequences (which may or may not include, without limitation, heterozygous mixtures of sequences) identified from samples, either containing some or all of the pathogen such as HIV nucleic acid isolated from plasma samples from a patient with the pathogen or containing other biological material, such as the patients own nucleic acid. To obtain these sequences, a lab technician may perform conventional amplification processes as part of the isolation procedure. Amplification of the target nucleic acid can be performed according to conventional amplification procedures. For example, it is possible to amplify a selected portion of a target nucleic acid using the polymerase chain reaction (PCR) technique as described in U.S. Pat. Nos. 4,683,194, 4683,195 and 4,683,202, using ligase chain reaction as described in U.S. Pat. No. 5,427,930, using bDNA amplification as described in U.S. Pat. No. 5,624,802 or using isothermal amplification as described in U.S. Pat. Nos. 5,409,818 and 5,554,516, all of which are incorporated herein by reference.

Sequencing of the target nucleic acid can be performed on the isolated and optionally amplified nucleic acids according to techniques known in the art. For example, sequencing may be performed using techniques based on the “chain termination” method described by Sanger et al, Proc. Nat. Acad. Sci. 74(12):5463-5467 (1977), and improvements thereto, for Example, in U.S. Pat. Nos. 5,821,058, 5,437,911, 6,083,699 and 6,214,555, all of which are incorporated herein by reference.

The characteristics may be provided to input 208 for determination of mutations in the sequence of the pathogen by difference identifier 210. Difference identifier 210 compares the characteristics of the pathogen received at input 208 with the similar characteristics of a “target pathogen” to identify differences between the characteristics of the pathogen received at input 208 and the target pathogen. The target pathogen may be a hypothetical version of the pathogen that is only used for comparison purposes and need not actually exist in nature. The characteristics of the target nucleic acid are stored, having been received via input 213, in target pathogen characteristic storage 212, which may be conventional memory or disk storage and may include conventional database software, such as the conventional Oracle 11i product commercially available from Oracle corporation of Redwood Shores, Calif., the conventional SQL Server product commercially available from Microsoft Corporation of Redmond, Wash., or any other similar database product.

For example, if the characteristics are a complete sequence of HIV RNA or DNA, or portions of these nucleic acids, the target nucleic acid may be the DNA or RNA of the conventional wildtype HIV-1 virus and the characteristics of this target may be portions or all of the nucleic acid sequence of this virus, including the identification of the location and name of the genes as well as the nucleotide sequence and such characteristics are used below, although the present invention applies to any characteristics and any type of target.

Once the characteristics are provided, the user signals difference identifier 210 to begin identifying differences. Difference identifier 210 compares the sequence or portions of sequences of the nucleic acid received at input 208 with the sequence or portions of sequences of the wildtype HIV-1 virus target nucleic acid stored in target pathogen characteristic storage 212. If portions of sequences are involved, difference identifier 210 may apply conventional homology algorithms, such as BLAST or its variants, to align the sequence or each portion received at input 208 with the sequence or one of the portions stored in target pathogen characteristic storage 212. As used herein, “BLAST” refers to the Basic Local Alignment Search Tool, described at http://www.ncbi.nlm.nih.gov/BLAST/tutorial/Altschul-1.html, and the information at this site is hereby incorporated by reference in its entirety.

The differences identified by difference identifier 210 may include a description of the characteristics that are different. For example, this description may include locations (e.g. codon position within a specific gene, together with the amino acid(s) detected at the location) on the nucleic acid of mutations between the HIV nucleic acid sequence or sequences isolated from the patient and received at input 208 and the wildtype virus sequence or sequences stored in target pathogen characteristic storage 212. The differences may include a number of inserted or deleted codons at each location or other information about differences of any characteristics of the pathogen received at input 208 with the characteristics of the target pathogen stored in target pathogen characteristic storage 212. Difference identifier 210 passes these differences to rules matcher/identifier 214 and signals rules matcher/identifier 214 to begin identifying applicable rules.

Upon receipt of the signal, rules matcher/identifier 214 accepts the differences from difference identifier 210 and determines if the differences match any of the criteria described in one or more rules stored in rules storage 216 via input 217. This determination may be made using conventional artificial intelligence techniques or programs, such as the conventional CLIPS system, version 6, available from the National Aeronautics and Space Administration (NASA), available at http://www.siliconvalleyone.com/clips.htm (and the information at this site is hereby incorporated by reference in its entirety), or other conventional techniques for matching rules to a particular set of facts. For each rule to which the differences received by rules matcher/identifier 214 correspond to one or more of the criteria in the rule, rules matcher/identifier 214 identifies the rule by storing an identifier of the rule into identified rule storage 220.

Each rule identifies one or more criteria that, if met, specify a level of resistance that may be expected if certain treatments corresponding to the rule are applied to the nucleic acid having the differences received by rules matcher/identifier 214. Each rule may have a number or other identifier. In one embodiment, the rules stored in rules storage 216 include the criteria specified on the “mutations involved” or “rules involved” line of each rule listed in Appendix A, along with the rule number listed for the rule in Appendix A. A criteria may include a description of the one or more mutations at specific locations, such as a specific location on a gene or may include a mutation (including a deletion or insertion of one or more codons) anywhere or any mutation at a location. A criteria may include the lack of a mutation anywhere or at a specific location, such as on a gene. A criteria may also include whether the criteria for one or more other rule has been satisfied or has not been satisfied, such as the criteria for rule 72 being that the criteria is met for any rule for which the drugs affected are saquinavir, indinavir or amprenavir (although the criteria for these rules, linked together by logic statements such as “or” may be used in place of the criteria of whether or not the criteria for another rule or rules has been satisfied or not satisfied). Any other condition relating to the nucleic acid, a different nucleic acid or conditions unrelated to the nucleic acid may be included in a rule, and such conditions may be input to the system with the characteristics of the nucleic acid.

As an example, the criteria for rule 1 from Appendix A would be stored in rules storage 216 as:

Rule 1: 2 or more of: M41L K65R D67N K70R L74V
Y115F L210W T215Y/F K219E/Q (Rule 1)

In one embodiment, rules matcher/identifier 214 allows the use of wild cards in the criteria of any rule. In one embodiment, a wild card can correspond to any number of deletions or insertions at a specific codon. In one embodiment, a specific number of insertions or a specific number of deletions may be specified by repeating the wildcard one more than that many times to indicate that an exact match of that number of deletions or insertions is the criteria for the rule. For example, a criteria for a rule could read “69XXX”, implying that the resistance effect in the rule corresponds to the existence of three codons (the one expected and two that have been inserted) at codon 69.

Rules matcher/identifier 214 applies the differences it receives to all of the criteria for all of the rules in rules storage 216 and identifies the rules having one or more criteria that apply to the differences it receives from difference identifier 210. Thus, if rules matcher/identifier 214 receives from difference identifier 210 an indication that 2 or more mutations from the wildtype virus exist in the HIV virus sequence or sequences received at input 208 at sequence locations M41L, K65R, D67N, K70R, L74V, Y115F, L210W, T215Y/F, or K219E/Q, rules matcher/identifier 214 will identify by storing in an entry in identified rule storage 220 an identifier for rule 1, such as “1”, in addition to identifiers of any other rules having criteria that correspond to the differences received. Each entry may correspond to an object, a record, or a row in a database and identified rule storage 220 may contain memory or disk storage and even a database such as those described above. In addition to storing the identifier of the rule, rules matcher/identifier 214 also stores in identified rule storage 220 an indication of the difference or differences that caused the rule to be identified (e.g. “M41L and K65R”) and associates the difference or differences with the identifier of the rule. Once rules matcher/identifier 214 identifies each rule, the identifier of the rule may be retrieved by a user from identified rule storage 220 using input/output 221, for example, by coupling it to a conventional input/output device such as a conventional keyboard/monitor/mouse combination (not shown) to allow the user to identify specific rules identified.

In one embodiment, each of the rules are assigned into groups of rules. Each of the rules assigned to a group are logically related to one another. In one embodiment, one group includes rules related to treatments involving reverse transcriptase inhibitors, and another group involves riles related to treatments involving protease inhibitors. In one embodiment, one or more groups contain two or more subgroups, and the rules are assigned to a group by assigning the rule to a subgroup in the group. In one embodiment, the subgroups of the reverse transcriptase inhibitor group include nucleoside reverse transcriptase inhibitors and nonnucleoside reverse transcriptase inhibitors. In one embodiment, the rules are assigned to groups and/or subgroups by storing an identifier of the group and/or subgroup with the rule in rules storage 216. In another embodiment, rules storage 216 is logically divided into areas assigned to each group or subgroup and each rule is assigned to the group or subgroup by storing it into the area assigned to the group or subgroup in rules storage 216.

For example, in Appendix A, when the rules are entered to rules storage 216, rules 1-71 are assigned to a reverse transcriptase inhibitor rules group, with rules 1-31 assigned to a nucleoside reverse transcriptase inhibitor rules subgroup, and rules 32-71 assigned to a nonnucleoside reverse transcriptase inhibitor rules subgroup. Rule 72 is assigned to a protease inhibitor rules group.

In one embodiment, rules need not be explicitly assigned into subgroups, as the criteria, treatments or other information related to each rule may be used by the elements of the system 200 that use the group and subgroup assignments as described below to determine the group and subgroup to which the rule would have been assigned. For example, the location on the reverse transcriptase gene for these mutations is implicit from the criteria of Rule 1.

In one embodiment, each rule corresponds to information such as one or more treatments and a level of resistance of the treatment or treatments corresponding to the rule that can be expected when any treatment corresponding to the rule is applied to treat the pathogen. In Appendix A, the level of resistance is noted as “Resistance Effect” and the treatment or treatments are noted as “Drugs Affected”, although other ways of noting the treatment and level of resistance may be used. In one embodiment, this other information for each rule, such as level of resistance and treatments for which that level of resistance is expected when the pathogen corresponds to the criteria, is stored in rules storage 216, associated with the identifier of the rule to which the treatment and level of resistance corresponds. When rules matcher/identifier 214 stores the identifier of the rule in each entry in identified rule storage 220, it may store such additional information in the entry in identified rules storage 220 with the identifier of the rule. In one embodiment, the criteria for the rule as well as the relevant differences corresponding to the criteria for the rule received from difference identifier 210 are also retrieved from rules storage 216 and stored by rules matcher/identifier 214 in the entry for the rule in identified rules storage 220 for each rule identifier stored therein.

In one embodiment the level of resistance stored in rules storage 216 corresponding to each rule may be any one of “Not Resistant”, “Possible Resistance” and “Resistance”, although other numbers of levels of resistance or descriptions may be used. For example, “Not Resistant” may not be used in one embodiment of the present invention. In one embodiment, “Possible Resistance” is used to signify that possible resistance may occur if the mutations detected have been associated with diminished virological response in some, but not all, patents or if the detected mutations have been associated with intermediate decrease in antiretroviral susceptibility in viral isolates. “Resistance” is used to signify the mutations in the rule have been associated with maximum reduction in susceptibility to the treatment. Treatments having this level of resistance may be appropriate for patients having few or no other treatment options. Other indicators related to resistance, such as susceptibility levels, may also be used by the present invention to mean similar or different indications of resistance. These other levels may include “partially resistant”. The level of resistance may be more continuous than a small number of discrete levels, and could for example, correspond to a larger number (e.g. 10, 50 or 100) of discrete levels, an integer or real number.

In one embodiment, some differences meeting the criteria of one or more rules may not be known to affect the resistance of the pathogen to a treatment, but treatment may be assisted by noting certain comments to the care provider when the presence of such differences are detected. In such embodiment, such rules are provided with a level of resistance as “Comment”, along with a comment to the care provider, such as is listed in Rule 71 of Appendix A, stored in rules storage 216 for use as described below. The comment may include a notification that a resistance effect of the difference is suspected but not known or another similar comment.

In one embodiment, the treatments corresponding to each rule stored in rules storage 216 may be one or more of any drug or combination of drugs or other forms of treatments. Drugs may include any antiviral compound where polymorphic changes in genomic content occurs, including the class of drugs known as reverse transcriptase inhibitors, integrase inhibitors, antifusion compounds or protease inhibitors, and may include any one or more of abacavir, tenofovir, didanosine, zalcitabine, lamivudine, stavudine, zidovudine, foscarnet, delavirdine, efavirenz, nevirapine, saquinavir, lopinavir with ritonavir, amprenavir, indinavir, nelfinavir and ritonavir, although other drugs and types of drugs may be used. In one embodiment, the treatments in treatment/supplemental information storage include at least three, five, seven, or ten of the reverse transcriptase inhibitors and protease inhibitors listed above. In one embodiment, when rules matcher/identifier 214 finishes identifying all rules corresponding to the differences it receives, rules matcher identifier 214 signals default resistance level manager 230.

Upon receipt of the signal, default resistance level manager 230 identifies and stores in default resistance level storage 232, for each of the treatments corresponding to any of the rules in rules storage 216, a default level of resistance. A default level of resistance is a level of resistance that is to be identified for each treatment if the differences between the characteristics of the target pathogen in target pathogen characteristic storage 212 and the characteristics of the pathogen received at input 208 do not correspond to any of the rules for that treatment. In one embodiment, the default level of resistance for each treatment is the lowest level of resistance or varying levels of resistance. In one embodiment, default resistance level manager 230 copies the default resistance levels from, or initializes and uses, a predefined list of available treatments and default resistance levels stored in default resistance level storage 232, received via input 231 coupled to a conventional keyboard/mouse.

In another embodiment, default resistance level manager 230 calculates the default resistance using a list of available treatments stored in default resistance level storage 232 via input 231. The default resistance is calculated by scanning the rules in rules storage 216 to determine whether each treatment corresponds to any of the treatments specified in the rules in rules storage 216. Default resistance level manager 230 calculates the default resistance level by selecting a treatment from the list, determining whether the treatment corresponds to a rule, if so, storing into default resistance level storage 232 a default resistance level for the treatment as “not resistant” and if not, storing into default resistance level storage 232 a default resistance of “insufficient evidence”. The use of “insufficient evidence” as a default level of resistance for a treatment instead of “no resistance” can inform the care provider that no rules are available to determine the level of resistance for the treatment, allowing the care provider to determine that system 200 is not providing any guidance whatsoever for the treatment. In one embodiment, comment rules are ignored for purposes of calculating a default resistance and in another embodiment, such rules are not ignored for this purpose.

In one embodiment, a combination of the above techniques may be used to determine the default resistance for each treatment, for example by storing a set of defaults as “insufficient evidence”, with default resistance level manager 230 copying the list into a set of default levels of resistance in default resistance level storage 232, then scanning the rules and replacing the default level of resistance as “not resistant” in the set of default values in default resistance level storage 232 for any treatment corresponding to a rule in rules storage 216. In one embodiment, when default resistance level manager 230 finishes identifying the default resistance levels for each of the treatments, default resistance level manager 230 signals high resistance level provider 222.

High resistance level provider 222 selects each of the treatments in default resistance level storage 232 and scans the treatments and levels of resistances corresponding to the rules stored in identified rule storage 220 and determines, for the currently selected treatment, the highest level of resistance for that treatment from the levels of resistance for that treatment corresponding to the entries in identified rule storage 220. In one embodiment, the “Comment” level of resistance is the lowest level of resistance, “Not Resistant” level is the second lowest level of resistance (if such a level exists), “Possible Resistance” is the second highest level of resistance and “Resistant” is the highest, but other embodiments may have different orders of levels of resistance, such as a swapping “Not Resistant” and “Resistant” in the hierarchy. For the highest level of resistance identified for the treatments corresponding to the entries in identified rule storage 220 by high resistance level provider 222, high resistance level provider 222 marks the entries in identified rule storage 220 with a description of the treatment or treatments for which the rule has been identified as having the highest level of resistance and marks the treatment in default resistance level storage 232 to indicate that at least one rule was identified for that treatment. In one embodiment, high resistance level provider 222 marks the treatment in default resistance level storage 232 with the identifier of the rule or rules that correspond to the highest resistance level for the treatment for use as described below.

If rules are arranged into groups and/or subgroups as described above, in one embodiment, high resistance level provider 222 only uses rules corresponding to the same group or the same group and subgroup as the treatment selected from default resistance level storage 232 when it identifies the highest resistance level for the treatment and determines whether any rule corresponding to the treatment was identified and stored in identified rule storage 220 to mark the treatment in default resistance level storage 232.

High resistance level provider 222 repeats the process described above for each treatment in default resistance level storage 232. When high resistance level provider 222 has identified the highest resistance level corresponding to each treatment in default resistance level storage 232, high resistance level provider 222 signals supplemental information provider 226.

In one embodiment, each rule corresponds to supplemental information. Supplemental information is information about the rule, the treatment or treatments corresponding to the rule, the criteria for the rule or the level of resistance corresponding to the rule. In one embodiment, the supplemental information may include a description of any publications from which the rule was derived. In one embodiment, the supplemental information may include a description of the type of research from which the rule was derived, for each of the treatments specified in the rule. The type of research may include any one of a first type corresponding to a plurality of large, independent virological response studies and supporting in vitro data, a second type corresponding to in vitro data and preliminary virological response data, a third type corresponding to in vitro data, and a fourth type corresponding to extrapolation of data. (In Appendix A, the type of research for each treatment in a rule is indicated by the superscripted Roman numerals adjacent to the name of the drug in the Drug(s) Affected portion of each rule.) In one embodiment, supplemental information is stored via input 225 in supplemental information storage 224 associated with the identifier of the rule to which the supplemental information corresponds. Supplemental information provider 226 scans the entries in identified rule storage 220, retrieves from treatment/supplemental information storage 224 the supplemental information corresponding to each rule identifier in each entry in identified rule storage 220, and provides it to each entry in identified rule storage 220 corresponding to the rule to which the supplemental information applies.

In one embodiment, the extrapolation of data from the fourth type above may be performed by researchers using indirect evidence of resistance effects provided by data from similar antiretroviral agents and/or mutation patterns or from unpublished data. Extrapolation of data may be performed where there are no in vitro or in vivo data regarding one or more mutations or patterns of mutations.

Some rules that do not have a level of resistance as “comment” may nevertheless have associated comments for that rule and these comments are stored in supplemental information storage 224 associated with the rule. Such comments are retrieved from supplemental information storage 225 and provided into the entry corresponding to the rule with which the comment is associated in identified rule storage 220 by supplemental information provider 226 either in one embodiment, for all of the rules corresponding to the identifiers stored in identified rule storage 220 and in another embodiment, for those rules marked as having the highest level of resistance for each treatment.

In one embodiment, supplemental information storage 224 contains comments about one or more treatments that are not specific to any rule. Supplemental information provider 226 retrieves and provides the text of such comments from supplemental information storage 224 and stores the comment in each entry in identified rule storage 220 that contains one of the treatments to which the comment relates.

In one embodiment, supplemental information provider 226 provides some or all of the supplemental information from supplemental information storage 224 for all of the rules having identifiers stored in identified rules storage 220, and in another embodiment, supplemental information provider 226 provides some or all of the supplemental information only for those rules marked by high resistance level provider 222 as corresponding to the highest level of resistance for each treatment, and in another embodiment, a combination of these techniques are used: some supplemental information is provided for the entry corresponding to the highest resistance level while other supplemental information is provided for a rule whether or not any treatments in the rule have the highest resistance level. When supplemental information provider 226 completes providing the supplemental information, it signals report generator 228.

In one embodiment, after receiving the signal, report generator 228 generates a report, describing for each treatment, or each treatment in each group and/or subgroup, either the default level of resistance for treatments for which no rule was identified in identified rule storage 230 or the highest level of resistance for that treatment for treatments for which at least one rule was identified in identified rule storage 230. All of the levels of resistance corresponding to rules identified for the treatment may be provided in the report by report generator 228 in addition to the highest level of resistance in other embodiments.

To perform this function, report generator 228 selects a treatment from default resistance level storage 232 and writes to a first column of the report a description of the treatment. Report generator 228 then determines whether any rules in identified rule storage 220 correspond to the treatment, or correspond to the treatment, group and/or subgroup. In one embodiment, report generator 228 performs this determination by checking whether any rule identifiers are stored with the default resistance level for the treatment or for the treatment, group and/or subgroup in default resistance level storage 232.

If report generator 228 determines that no rule in identified rule storage 220 corresponds to the selected treatment, report generator 228 provides as a second column of the report a description of the default resistance level for the treatment. If report generator 228 determines that at least one rule in identified rule storage 220 corresponds to the treatment, report generator 228 provides as the second column of the report the highest level of resistance. Report generator 228 repeats this process for each treatment stored in default resistance level storage 232, with each treatment selected corresponding to a different row of the report.

In one embodiment, report generator 228 performs the process described above for all treatments in one group before performing the process for treatments in another group. With each group, the process described above is performed for all treatments in one subgroup before the process is performed for the treatments in another subgroup. In doing so, report generator 228 thus organizes the report by group and subgroup.

In one embodiment, report generator 228 provides header or footer information in the report each time the group or subgroup changes. In one embodiment, header information for a group includes the indications of all of the differences stored in identified rule storage 220 for the rules corresponding to the group, with duplicate entries removed. This allows a care provider to identify the differences that corresponded to any rules for the group. In one embodiment, header information for a group includes a description of the group, unless there are subgroups in the group, in which case report generator 228 provides header information for each subgroup that includes a description of the subgroup.

In one embodiment, for each treatment, all levels of resistance corresponding to the entries in identified rule storage 220 may be provided by report generator 228 in addition to the highest level of resistance, and the highest level of resistance may be marked when all of the levels corresponding to entries in identified rule storage 220 are provided. Each level of resistance is marked with the type or types described above corresponding to the level of resistance for the treatment. This allows a care provider to see all of the levels of resistance identified, but easily spot the highest level for each treatment and use his experience and judgment in determining which level of resistance to accept for each treatment.

In one embodiment, the report generated by report generator 228 is color coded: each level of resistance is displayed using a color, which may be distinctive from the colors of other levels of resistance. In one embodiment, “Resistance” is displayed in red, “Possible Resistance” is displayed in orange and “No Evidence of Resistance” is displayed in green, although other colors may be used.

In one embodiment, report generator 228 generates a mutation details section of the report after it writes the first and second columns of the report described above. The mutation details section may be written after a page break to ensure that it begins at the top of a page. In this section, report generator 228 selects from default resistance level storage 232 a group having no subgroups, or a subgroup within a group, and for each rule corresponding to the selected group or subgroup that is stored in identified rules storage 220 (or for the rules marked as corresponding to the highest level of resistance for a treatment as described above), report generator 228 provides a text description of the differences that caused the identifier of the rule to be included in identified rule storage 220 (which were stored by rules matcher/identifier 214 as indications of the differences as described above) and description of the level of resistance and treatment. So for example, report generator 220 might describe rule 1 in the mutation details section as “M41L K65R D67N indicate possible resistance to abacavir” if differences at location M41L K65R D67N were the differences that caused rules matcher/identifier 214 to identify the rule. In one embodiment, report generator 228 also provides supplemental information for the rule, for example, by providing an identifier of the type of research for each of the treatments corresponding to the rule (or an identifier of the type of research for the treatments for which the rule is marked as corresponding to the highest level of resistance as described above). In one embodiment, the Roman numerals described above may be used as the indicators of the type of research, with a key provided by report generator 228 as described in more detail below.

Report generator 220 also provides header information for each group or subgroup in a manner similar to that provided for the first and second columns described above. Report generator 228 may also provide a key following the mutation details section that describes the terms used in the levels of resistance and the identifiers of the type of research.

In one embodiment, report generator 228 is coupled to difference identifier 210. In such embodiment, the characteristics of the nucleic acid (such as the nucleic acid sequence of the patient's HIV virus) and the pertinent differences identified are retrieved by report generator 228 and included in the report. In such embodiment, patient identification information may be input to difference identifier 210 via input 208 and such information is included in the report produced by report generator 228.

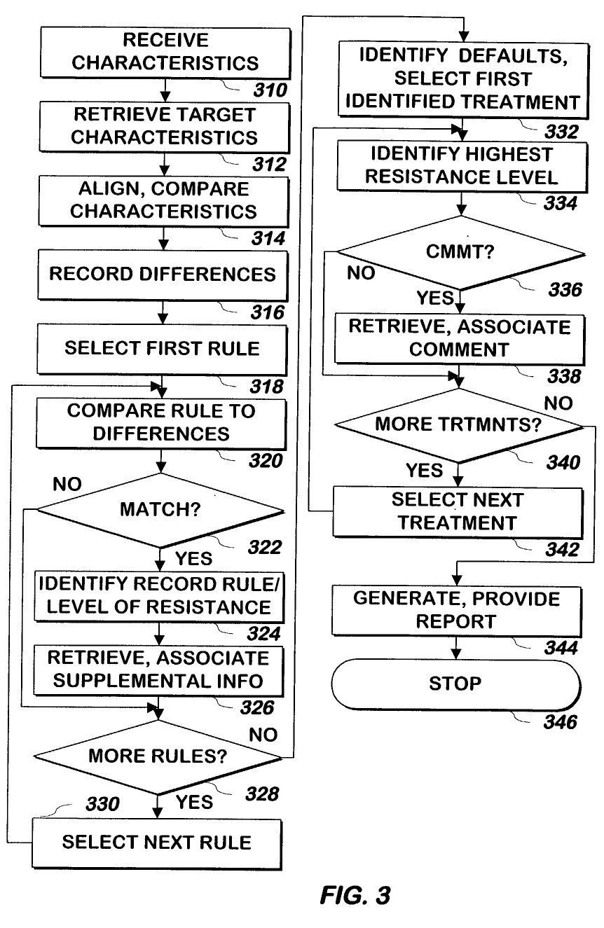

Referring now to FIG. 3, a method of analyzing resistance of a pathogen to one or more treatments is shown according to one embodiment of the present invention. Characteristics of a nucleic acid, such as the nucleic acid of the pathogen are received 310 and characteristics of a target nucleic acid are received 312 as described above. If necessary, the characteristics received in steps 310 and 312 are aligned 314, for example, using conventional homology techniques and the characteristics received in step 310 and 312 are compared 314. Differences between the characteristics are recorded 316 and a first rule is selected 318.

Differences specified by the criteria in the rule selected are compared with the differences recorded in step 316 and if a match occurs 322, the an identifier of rule (or the criteria or the level of resistance and the treatments corresponding to the rule, or all of these) is identified or recorded 324 as described above and the method continues at step 326 and otherwise 322, the method continues at step 328. At step 326, supplemental information corresponding to the selected rule is retrieved, which may include the treatment, level of resistance, type of study supporting the rule, comments from the rule and other information as described herein, and the supplemental information is associated with the rule. The method continues at step 328.

At step 328, if there are more rules that have not been selected 328, the next rule is selected 330 and the method continues at step 320 using that selected rule, and otherwise 328, the method continues at step 332.

At step 332, default levels of resistances are identified as described above and a first treatment corresponding to the rule or level of resistance for the treatments identified in step 324 is selected and the highest level of resistance for that treatment from the levels identified (or corresponding to the rules identified) in one or more iterations of step 324 is identified 334.

If a comment for the treatment exists, the comment is retrieved and associated with each rule identified in step 324 that specifies the treatment 338 and the method continues at step 340 and otherwise 336, the method continues at step 340.

If there are more treatments corresponding to the rules or levels of resistance for the treatments identified in step 324 340, another treatment is selected 342 and the method continues at step 332 using that selected treatment, and otherwise 348, the method continues at step 352. Otherwise 340, the method continues at step 344 where a report is generated 344 as described herein and the method terminates 346 or begins again at step 310.

[image]

[image]

[image]

[image]

[image]

[image]

[image]

[image]

[image]

[image]

[image]

[image]

[image]

[image]

[image]

[image]

[image]

[image]

[image]

What is claimed is:

1. A method of identifying for a pathogen at least one level of resistance to each of a plurality of treatments, comprising:
receiving at least one difference between a nucleic acid and a target nucleic acid;
receiving, for each of the plurality of treatments, a plurality of rules, each rule comprising an indication of a level of resistance corresponding to a set of at least one criteria, each of the at least one criteria comprising at least one difference between a subject nucleic acid and the target nucleic acid;
attempting to match each of the set of at least one difference in each of the criteria for the plurality of rules to the differences received; and
identifying at least one level of resistance for at least a first one of the plurality of treatments responsive to the attempting to match step.

2. The method of claim 1, additionally comprising, for each treatment corresponding to the at least one level of resistance identified, providing a highest level of resistance identified for said treatment.

3. The method of claim 2, additionally comprising, for each treatment corresponding to the at least one level of resistance identified, providing levels of resistance identified other than the highest level of resistance identified.

4. The method of claim 1, additionally comprising, for each treatment corresponding to the at least one level of resistance identified, providing supplemental information about at least one of at least one selected from the at least one level of resistance identified, and the plurality of treatments corresponding to the at least one level of resistance identified.

5. The method of claim 4, wherein the supplemental information comprises a type of research indicating said level of resistance, the type comprising at least one of:
a first type corresponding to a plurality of large, independent virological response studies and supporting in vitro data;
a second type corresponding to in vitro data and preliminary virological response data;
a third type corresponding to in vitro data; and
a fourth type corresponding to extrapolation of data.

6. The method of claim 5 wherein the type of at least one study comprises a plurality of the first type, the second type, the third type and the fourth type.

7. The method of claim 6, wherein the type of at least one study comprises the fourth type

8. The method of claim 1, wherein the receiving at least one difference step comprises:
receiving a description of at least a portion of the nucleic acid and a description of at least a portion of the target nucleic acid; and
identifying at least one difference between a specific location of each of said portions.

9. The method of claim 8, wherein each of the at least one difference identified between a specific location of each of said portions comprises at least one mutation of a biological sequence.

10. The method of claim 1 wherein the nucleic acid is a nucleic acid of the pathogen.

11. The method of claim 10 wherein the pathogen is a bacteria.

12. The method of claim 10 wherein the pathogen is a virus.

13. The method of claim 12 wherein the virus comprises Human Immunodeficiency Virus.

14. The method of claim 1 wherein at least some of the plurality of treatments comprise at least one antiviral compound.

15. The method of claim 14 wherein the at least one antiviral compound comprises at least ten of abacavir, tenofovir, didanosine, zalcitabine, lamivudine, stavudine, zidovudine, foscarnet, delavirdine, efavirenz, nevirapine, saquinavir, lopinavir with ritonavir, amprenavir, indinavir, nelfinavir and ritonavir.

16. The method of claim 1 wherein the plurality of treatments comprise at least ten treatments.

17. A system for identifying for a pathogen at least one level of resistance to each of a plurality of treatments comprising:
a rules matcher having a first input operatively coupled for receiving at least one difference between a nucleic acid and a target nucleic acid, and a second input operatively coupled for receiving a plurality of rules, each comprising at least one criteria for a match, the criteria comprising at least one difference between the target nucleic acid and at least one subject nucleic acid, the rules matcher for attempting to match the differences received at the first input with at least one criteria for the match corresponding to each of the plurality of rules and for providing at an output an identifier of each rule for which the at least one difference received at the first input matched at least one of the at least one criteria; and
a supplemental information provider having a first input coupled to the rules matcher output for receiving the identifiers of the rule and a second input operatively coupled for receiving, for each of the rules having the identifiers received at the rules matcher first input, at least one treatment and at least one resistance level, the supplemental information provider for providing at an output the at least one treatments the at least one resistance levels corresponding to the at least one identifier received at the supplemental information provider second input.

18. The system of claim 17, additionally comprising a report generator, having an input coupled to the supplemental information provider output, the report generator for, for each treatment received at the report generator input, providing at an output a highest level of resistance corresponding to said treatment.

19. The system of claim 18, wherein the report generator is additionally for, for each treatment corresponding received at the report generator input, providing at the report generator output the levels of resistance received at the report generator input other than the highest level of resistance identified.

20. The system of claim 17, wherein the supplemental information provider is additionally for, for each treatment corresponding to the at least one level of resistance identified, providing at the supplemental information provider output supplemental information about at least one of at least one selected from the at least one level of resistance identified, and the plurality of treatments corresponding to the at least one level of resistance identified.

21. The system of claim 20, wherein the supplemental information comprises a type of research indicating said level of resistance, the type comprising at least one of:
a first type corresponding to a plurality of large, independent virological response studies and supporting in vitro data;
a second type corresponding to in vitro data and preliminary virological response data;
a third type corresponding to in vitro data; and
a fourth type corresponding to extrapolation of data.

22. The system of claim 21 wherein the type of at least one study comprises a plurality of the first type, the second type, the third type and the fourth type.

23. The system of claim 22, wherein the type of at least one study comprises the fourth type

24. The system of claim 17, additionally comprising a difference identifier comprising a first input operatively coupled for receiving a description of at least a portion of the nucleic acid and a second input operatively coupled for receiving a description of at least a portion of the target nucleic acid, the difference identifier for identifying and providing a description at an output of at least one difference between a specific location of each of said portions.

25. The system of claim 24, wherein each of the at least one difference identified between a specific location of each of said portions comprises at least one mutation of a biological sequence.

26. The system of claim 17 wherein the nucleaic acid comprises a nucleic acid of the pathogen.

27. The system of claim 26 wherein the pathogen is a bacteria.

28. The system of claim 26 wherein the pathogen is a virus.

29. The system of claim 28 wherein the virus comprises Human Immunodeficiency Virus.

30. The system of claim 17 wherein at least some of the plurality of treatments comprise at least one antiviral compound.

31. The system of claim 30 wherein the at least one antiviral compound comprises at least ten of Abacavir, Tenofovir, Didanosine, Zalcitabine, Lamivudine, Stavudine, Zidovudine, Foscarnet, Delavirdine, Efavirenz, Nevirapine, Saquinavir, Lopinavir with ritonavir, Amprenavir, Indinavir, Nelfinavir and Ritonavir.

32. The method of claim 17 wherein the plurality of treatments comprise at least ten treatments.

33. A computer program product comprising a computer useable medium having computer readable program code embodied therein for identifying for a pathogen at least one level of resistance to each of a plurality of treatments, the computer program product comprising computer readable program code devices configured to cause a computer to:
receive at least one difference between a nucleic acid and a target nucleic acid;
receive, for each of the plurality of treatments, a plurality of rules, each rule comprising an indication of a level of resistance corresponding to a set of at least one criteria, each of the at least one criteria comprising at least one difference between a subject nucleic acid and the target nucleic acid;
attempt to match each of the set of at least one difference in each of the criteria for the plurality of rules to the differences received; and
identify at least one level of resistance for at least a first one of the plurality of treatments responsive to the attempting to match step.

34. The computer program product of claim 33, additionally comprising computer readable program code devices configured to cause a computer to, for each treatment corresponding to the at least one level of resistance identified, provide a highest level of resistance identified for said treatment.

35. The computer program product of claim 34, additionally comprising computer readable program code devices configured to cause a computer to, for each treatment corresponding to the at least one level of resistance identified, provide levels of resistance identified other than the highest level of resistance identified.

36. The computer program product of claim 33, additionally comprising computer readable program code devices configured to cause a computer to, for each treatment corresponding to the at least one level of resistance identified, provide supplemental information about at least one of at least one selected from the at least one level of resistance identified, and the plurality of treatments corresponding to the at least one level of resistance identified.

37. The computer program product of claim 36, wherein the supplemental information comprises a type of research indicating said level of resistance, the type comprising at least one of:
a first type corresponding to a plurality of large, independent virological response studies and supporting in vitro data;
a second type corresponding to in vitro data and preliminary virological response data;
a third type corresponding to in vitro data; and
a fourth type corresponding to extrapolation of data.

38. The computer program product of claim 37 wherein the type of at least one study comprises a plurality of the first type, the second type, the third type and the fourth type.

39. The computer program product of claim 38, wherein the type of at least one study comprises the fourth type

40. The computer program product of claim 33, wherein the computer readable program code devices configured to cause a computer to receive at least one difference comprise computer readable program code devices configured to cause a computer to:
receive a description of at least a portion of the nucleic acid and a description of at least a portion of the target nucleic acid; and
identify at least one difference between a specific location of each of said portions.

41. The computer program product of claim 40, wherein each of the at least one difference identified between a specific location of each of said portions comprises at least one mutation of a biological sequence.

42. The computer program product of claim 33 wherein the nucleic acid comprises a nucleic acid of the pathogen.

43. The computer program product of claim 42 wherein the pathogen is a bacteria.

44. The computer program product of claim 42 wherein the pathogen is a virus.

45. The computer program product of claim 44 wherein the virus comprises Human Immunodeficiency Virus.

46. The computer program product of claim 33 wherein at least some of the plurality of treatments comprise at least one of antiviral compound.

47. The computer program product of claim 46 wherein the at least one antiviral compound comprises at least ten of Abacavir, Tenofovir, Didanosine, Zalcitabine, Lamivudine, Stavudine, Zidovudine, Foscarnet, Delavirdine, Efavirenz, Nevirapine, Saquinavir, Lopinavir with ritonavir, Amprenavir, Indinavir, Nelfinavir and Ritonavir.

48. The computer program product of claim 33 wherein the plurality of treatments comprise at least ten treatments.

49. The method of claim 1 additionally comprising:
identifying a default level of resistance for each of at least one of the plurality of treatments; and
providing at least one of the default levels identified.

50. The method of claim 49 wherein the identifying step is responsive to an existence of a rule corresponding to said treatment.
